(19) United States
(12) Patent Application Publication
(10) Pub. No.: US 2006/0093658 A1
(43) Pub. Date: May 4, 2006

(54) Apparatus and method for transdermal delivery of desmopressin

(76) Inventors: Gayatri Sathyan, San Jose, CA (US); Richard Weyers, Los Altos, CA (US); Peter Daddona, Menlo Park, CA (US); Peter Staehr, Mountain View, CA (US); Suneel Gupta, Sunnyvale, CA (US); Mahmound Ameri, Fremont, CA (US); Michel J.N. Cormier, Mountain View, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/259,010

(22) Filed: Oct. 25, 2005

Related U.S. Application Data
(60) Provisional application No. 60/622,467, filed on Oct. 26, 2004.

(51) Int. Cl. A61F 13/02; A61M 31/00

(52) U.S. Cl. 424/448; 604/500

(57) ABSTRACT

An apparatus and method for transdermally delivering desmopressin comprising a delivery system having a microprojection member (or system) that includes a plurality of microprojections (or array thereof) that are adapted to pierce through the stratum corneum into the underlying epidermis layer, or epidermis and dermis layers. In one embodiment, the desmopressin is contained in a biocompatible coating that is applied to the microprojection member.

### CROSS-REFERENCE TO RELATED APPLICATIONS

This application claims the benefit of U.S. Provisional Application No. 60/622,467, filed Oct. 26, 2004.

### FIELD OF THE PRESENT INVENTION

The present invention relates generally to transdermal agent delivery systems and methods. More particularly, the invention relates to an apparatus and method for transdermal delivery of desmopressin.

### BACKGROUND OF THE INVENTION

Active agents (or drugs) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent intravenously or subcutaneously, while assuring no modification of the agent during administration, is a difficult, inconvenient, painful and uncomfortable procedure that sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

It is well documented that the stratum corneum, the outermost layer of the skin, constitutes an impermeable barrier to hydrophilic or high molecular weight drugs, such as desmopressin. These molecules can only be delivered into or through the skin, if the barrier function of the stratum corneum is disrupted by any of a number of available methods. One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue Pat. No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

As is well known in the art, enuresis is a condition where involuntary voiding of urine occurs at least twice a month in a child age five or older. Children vary markedly in the age at which they are physiologically ready to awaken from sleep aware of the need to urinate. This hinders their ability to hold their urine throughout the night. If the child has never been totally dry for a year, the condition is known as primary enuresis. Eighty-percent of children who wet their bed suffer from primary enuresis. Secondary enuresis is when a child has had a dry period of at least a year before the appearance of the problem. The child invariably urinates during the first third of the night and remembers nothing of the occurrence. Although in 1 percent of cases, enuresis continues into adulthood, most children are continent by adolescence. Aside from wet pajamas, enuresis itself causes no direct impairment of the child's life, but social ostracism by peers (at sleepovers and camp, for example), and anger and rejection by parents can damage self-esteem. Enuresis seems to also occur frequently in late life. The prevalence of incontinence increases with age and affects more women than men, until after age 80, when men are equally affected. Of persons 65 years and older, 15% to 30% in the community and up to 50% in long-term care are incontinent.

Desmopressin is a potent synthetic peptide hormone, more specifically a synthetic analog of arginine vasopressin (AVP), that is used chiefly for treatment of enuresis in young children, as well as for diabetes insidipus, Hemophilia A and von Willebrand's Disease (Type I) prior to surgery, and for trauma-induced injuries. Desmopressin has hydro-osmotic effects similar to the native hormone, with much reduced vasopressor effects. It has selective antidiuretic activity.

Despite the efficacy of desmopressin in treating enuresis, there are several drawbacks and disadvantages associated with the disclosed prior art methods of delivering desmopressin, particularly, via subcutaneous injection. A major drawback is that subcutaneous injection is a difficult and uncomfortable procedure, which often results in poor patient compliance, especially with children. While a more acceptable route of administration is oral or nasal administration, desmopressin, however, is a 1100 Da molecule that is typically taken in doses of 1 to 20 μg, and shows variable and low oral and nasal bioavailability (0.1 and 3.4%, respectively). A more acceptable route of administration, therefore, with potentially good bioavailability could be offered by transdermal delivery.

It would thus be desirable to provide an agent delivery system that facilitates minimally invasive administration of desmopressin. It would further be desirable to provide an agent delivery system that provides a pharmacokinetic profile of the desmopressin similar to that observed following subcutaneous administration.

It is therefore an object of the present invention to provide a transdermal agent delivery apparatus and method that provides intracutaneous delivery of desmopressin to a patient.

It is another object of the invention to provide a transdermal agent delivery apparatus and method that provides a pharmacokinetic profile of desmopressin agent similar to than that observed following intravenous or subcutaneous administration.

It is another object of the invention to provide a transdermal agent delivery apparatus and method that provides pharmacologically active blood concentration of desmopressin for a period of up to eight hours.

It is another object of the invention to provide desmopressin formulations for intracutaneous delivery to a patient.

It is another object of the present invention to provide a transdermal agent delivery apparatus and method that includes microprojections coated with a biocompatible coating that includes desmopressin.

### SUMMARY OF THE INVENTION

In accordance with the above objects and those that will be mentioned and will become apparent below, the apparatus and method for transdermally delivering desmopressin in accordance with this invention generally comprises a delivery system having a microprojection member (or system) that includes a plurality of microprojections (or array thereof) that are adapted to pierce through the stratum corneum into the underlying epidermis layer, or epidermis and dermis layers. In a preferred embodiment, the microprojection member includes a biocompatible coating having desmopressin disposed therein.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as a polymeric material. Alternatively, the microprojection member can be coated with a non-conductive material, such as Parylene®, or a hydrophobic material, such as Teflon® silicon or other low energy material.

The coating formulations applied to the microprojection member to form solid biocompatible coatings can comprise aqueous and non-aqueous formulations. Preferably, the coating formulations include desmopressin, which can be dissolved within a biocompatible carrier or suspended within the carrier.

The present invention is directed to desmopressin which is a synthetic analog of vasopressin, a peptide hormone secreted from the posterior pituitary. Arginine vasopressin is the form of the peptide found in humans, while lysine desmopressin is the porcine form. It should be understood that the present invention is intended to not only cover desmopressin, but also arginine vasopressin, and other analogs of vasopressin, and all other active fragments, degradation products, salts and simple derivatives and combinations thereof of desmopressin and/or arginine vasopressin or other vasopressin analogs. Reference in this specification to desmopressin should be understood to also include reference to arginine vasopressin, and other analogs of vasopressin, and all other active fragments, degradation products, salts and simple derivatives and combinations thereof of desmopressin and/or arginine vasopressin or vasopressin analogs.

Examples of pharmaceutically acceptable desmopressin salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, desmopressin is present in the coating formulation at a concentration in the range of approximately 1-30 wt. %.

More preferably, the amount of desmopresssin contained in the solid biocompatible coating (i.e., microprojection member or product) is in the range of approximately 1 μg-1000 μg, even more preferably, in the range of approximately 10-100 μg.

Also preferably, the pH of the coating formulation is below approximately pH 8. More preferably, the coating formulation has a pH in the range of approximately pH 2-pH 8. Even more preferably, the coating formulation has a pH in the range of approximately pH 3-pH 6.

In certain embodiments of the invention, the viscosity of the coating formulation that is employed to coat the microprojections is enhanced by adding low volatility counterions. In one embodiment, desmopressin has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

Another preferred embodiment is directed to a viscosity-enhancing mixture of counterions, wherein the desmopressin has a positive charge at the formulation pH and at least one of the counterion comprises an acid having at least two acidic pKas. The other counterion comprises an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

In the noted embodiments of the invention, the amount of counterion is preferably sufficient to neutralize the charge of the desmopressin. In such embodiments, the amount of the counterion or mixture of counterions is preferably sufficient to neutralize the charge present on the agent at the pH of the formulation. In additional embodiments, excess counterion (as the free acid or as a salt) is added to the peptide to control pH and provide adequate buffering capacity.

In another preferred embodiment, desmopressin and the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve a viscosity in the range of approximately 20-200 cp.

In a preferred embodiment of the invention, the viscosity-enhancing counterion comprises an acidic counterion, such as a low volatility weak acid that exhibits at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment, the counterion comprises a strong acid that exhibits at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions, wherein at least one of the counterion comprises a strong acid and at least one of the counterion comprises a low volatility weak acid.

Another preferred embodiment is directed to a mixture of counterions, wherein at least one of the counterion comprises a strong acid and at least one of the counterion comprises a weak acid having a high volatility and exhibiting at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is preferably present in an amount that is sufficient to neutralize the positive charge present on desmopressin at the pH of the formulation. In an additional embodiment, an excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can comprise sequestering agents, such sodium citrate, citric acid, EDTA (ethylene-dinitrilo-tetraacetic acid) or free radical scavengers, such as ascorbic acid, methionine, sodium ascorbate and the like. Presently preferred antioxidants comprise EDTA and methionine.

In the noted embodiments of the invention, the concentration of the antioxidant is preferably in the range of approximately 0.01-20 wt. % of the coating formulation. More preferably, the concentration of the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan lauratealkoxylated alcohols, such as laureth-4 and polyoxyethylene castor oil derivatives, such as Cremophor EL®.

In the noted embodiments of the invention, the concentration of the surfactant is preferably in the range of approximately 0.01-20 wt. % of the coating formulation. Preferably, the concentration of the surfactant is in the range of approximately 0.05-1 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethyl-methacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and inulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose and the like.

Preferably, the concentration of the stabilizing agent in the coating formulation is at a ratio of approximately 0.1-2.0:1 with respect to desmopressin, more preferably, approximately 0.25-1.0:1 with respect to desmopressin.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation. methacrylate), In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

In accordance with one embodiment of the invention, the method for delivering desmopressin to a subject comprises (i) providing a microprojection member having a plurality of stratum corneum-piercing microprojections, the microprojection member having a biocompatible coating disposed thereon that includes desmopressin, (ii) applying the microprojection member to a skin site on the subject, whereby the microprojections pierce the stratum corneum and deliver desmopressin to the subject.

Preferably, the coated microprojection member is applied to the skin site via an impact applicator.

Also preferably, the coated microprojection member is preferably left on the skin site for a period lasting from 5 seconds to 24 hours. Following the desired wearing time, the microprojection member is removed. In some embodiments, wherein desmopressin is in the range of approximately 1 μg-1000 μg of the biocompatible coating.

Further, the pharmacokinetic profile of the transdermally delivered desmopressin is preferably at least similar to the pharmacokinetic profile observed following intravenous or subcutaneous delivery. Depending upon the indication being treated, a bolus delivery or pulsatile delivery can be selected.

In the methods of the invention, transdermally delivered desmopressin preferably exhibits rapid on-set of biological action. Also preferably, transdermal delivery of a desmopressin exhibits sustained biological action for a period of up to 10 hours.

In one embodiment, the transdermally delivered desmopressin and the biocompatible coating comprises a dose of desmopressin in the range of approximately 10-100 μg dose, wherein delivery of desmopressin results in a plasma Cₘₐₓ of at least 50 pg/mL after one application.

The invention also comprises a method of improving the pharmacokinetics of a transdermally delivered desmopressin comprising providing a microprojection member having a plurality of stratum corneum-piercing microprojections, the microprojection member having a biocompatible coating disposed thereon that includes desmopressin and applying the microprojection member to a skin site on the subject, whereby the microprojections pierce the stratum corneum and deliver the desmopressin the subject so that delivery of the desmopressin has improved pharmacokinetics compared to the pharmacokinetics characteristic of intravenous or subcutaneous delivery.

In the noted embodiments, the improved pharmacokinetics can comprise increased bioavailability of the desmopressin. The improved pharmacokinetics can also comprise increased in Cₘₐₓ. Further, the improved pharmacokinetics can comprise decreased Tₘₐₓ. The improved pharmacokinetics can further comprise an enhanced absorption rate of desmopressin.

The apparatus and method of the invention can thus be employed safely and effectively in the treatment of osteoporosis and bone fractures.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a concentration profile, according to the invention;

FIG. 2 is a perspective view of a portion of one example of a microprojection member, according to the invention;

FIG. 3 is a perspective view of the microprojection member shown in FIG. 2 having a coating deposited on the microprojections, according to the invention;

FIG. 4 is a side sectional view of a microprojection member having an adhesive backing, according to the invention;

FIG. 5 is a side sectional view of a retainer having a microprojection member disposed therein, according to the invention;

FIG. 6 is a perspective view of the retainer shown in FIG. 4;

FIG. 7 is an exploded perspective view of an applicator and retainer, according to the invention;

FIG. 8: Scanning electron microscopy of a microneedle array coated with 80 ug desmopressin per array. General view (a). Front view of one microneedle (b). Top view of one microneedle (c). Side view of a row of microneedles (d). Bar=1 mm (a), Bar=50 um (b, c, and d).

FIG. 9: Microneedle array delivery system. The patch comprising the coated microneedle array affixed to an adhesive backing is illustrated in panel a. The patch loaded on the disposable retainer ring and the reusable applicator are illustrated in panel b.

FIG. 10: Depth of penetration of uncoated microneedle arrays (Control) and arrays coated with the indicated amounts of desmopressin following application to HGPs.

FIG. 11: Mass balance (ug (a), % of the loading dose (b)) of desmopressin delivered for 5 or 15 min from microneedle arrays coated with three doses of desmopressin. Desmopressin present on the skin surface and remaining on the microprojections following array removal as well as the desmopressin systemic delivery extrapolated from urinary excretion data were used to calculate the total amounts or the total percentages recovered.

FIG. 12: Comparison of desmopressin serum concentrations following administration of desmopressin by IV (11 ug) or coated microneedle array (MFLX) (82 ug) administration of desmopressin. Time 0 indicates the beginning of drug administration or injection. The microneedle array wearing time was 5 min.

FIG. 13: Mean desmopressin concentrations-time profiles.

FIG. 14: presents the mean (SD) plasma factor VIII concentration-time profiles following IV and MFLX treatments.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### DEFINITIONS

The term “transdermal”, as used herein, means the delivery of an agent into and/or through the skin for local or systemic therapy.

The term “transdermal flux”, as used herein, means the rate of transdermal delivery.

The terms “pulsatile delivery profile” and “pulsatile concentration profile”, as used herein, mean a post administration increase in blood serum concentration of desmopressin from a baseline concentration to a concentration in the range of approximately 50-1000 pg/mL in a period ranging from 1 min. to 4 hr., wherein Cₘₐₓ is achieved, and a decrease in blood serum concentration from Cₘₐₓ to the baseline concentration in a period ranging from 1-10 hrs. after Cₘₐₓ has been achieved.

As discussed in detail herein, in one embodiment of the invention, the noted pulsatile delivery profile is reflected (or evidenced) by a curve of desmopressin concentration in the host's blood serum versus time having an area under the curve (AUC) in the range of approximately 100-5000 h·pg/mL and a Cₘₐₓ in the range of approximately 50-1000 pg/mL for a microprojection member nominally containing 5-100 μg desmopressin.

The term “co-delivering”, as used herein, means that a supplemental agent(s) is administered transdermally either before desmopressin is delivered, before and during transdermal flux of desmopressin, during transdermal flux of desmopressin, during and after transdermal flux of desmopressin, and/or after transdermal flux of desmopressin. Additionally, two or more forms of desmopressin may be formulated in the coatings and/or formulations, resulting in co-delivery of desmopressin.

Examples of suitable desmopressin salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

It is to be understood that more than one active ingredient can be incorporated into the agent source, reservoirs, and/or coatings of this invention, and that the use of the term “desmopressin” as the active ingredient in no way excludes the use of two or more active agents.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

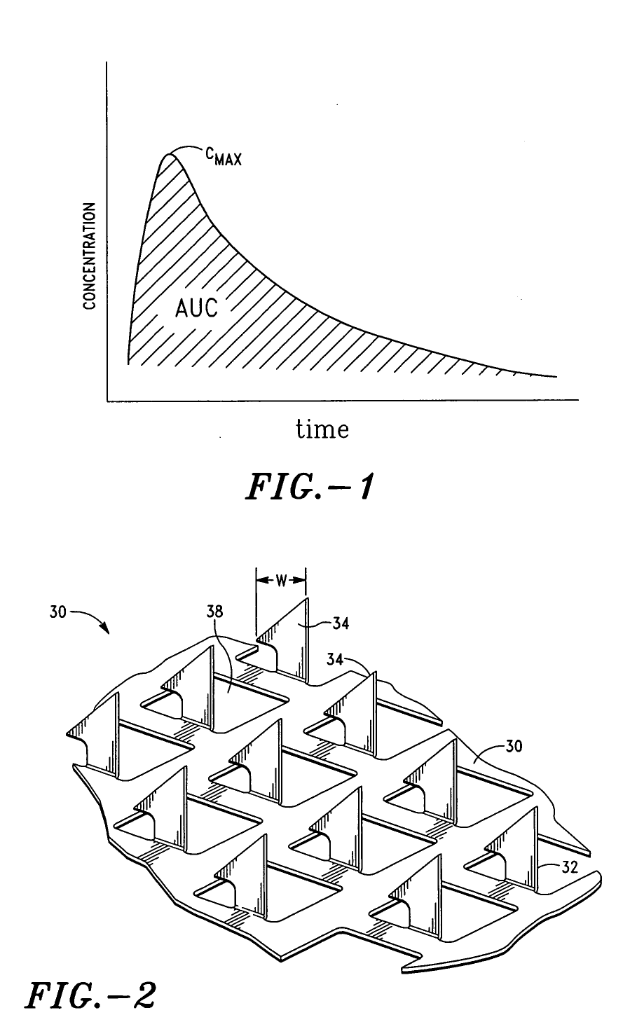

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

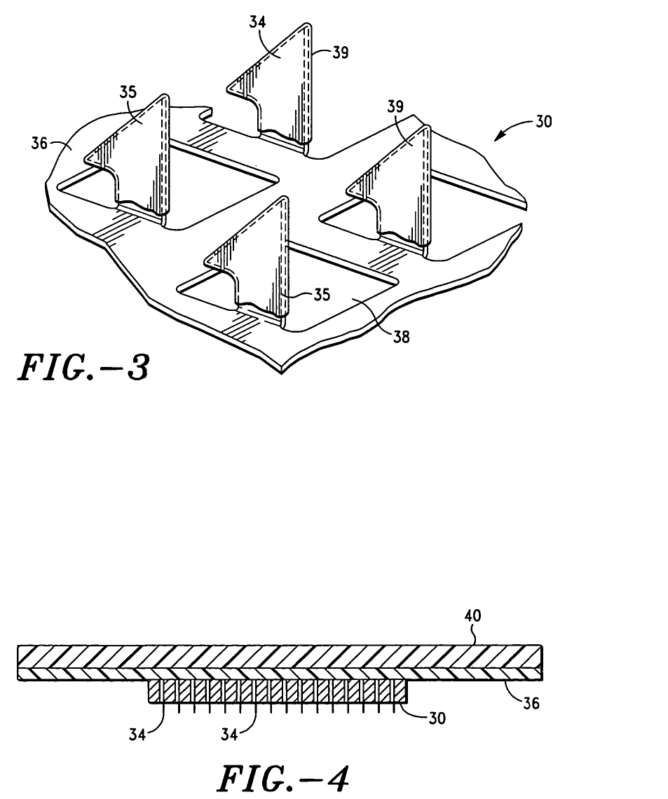

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. Preferably, the coating formulation includes desmopressin, which can be in solution or suspension in the formulation.

The term “biocompatible coating” and “solid coating”, as used herein, is meant to mean and include a “coating formulation” in a substantially solid state.

As indicated above, the present invention generally comprises a delivery system including microprojection member (or system) having a plurality of microprojections (or array thereof) that are adapted to pierce through the stratum corneum into the underlying epidermis layer, or epidermis and dermis layers.

As discussed in detail herein, a key advantage of the present invention is that the delivery system delivers desmopressin to a mammalian host, particularly, a human patient, whereby desmopressin in the patient's serum after administration exhibits a preferred pulsatile concentration profile. The delivery system is further amenable to self-administration of a 20 μg bolus dose of desmopressin at least once daily.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet, which in the noted embodiment includes openings 38.

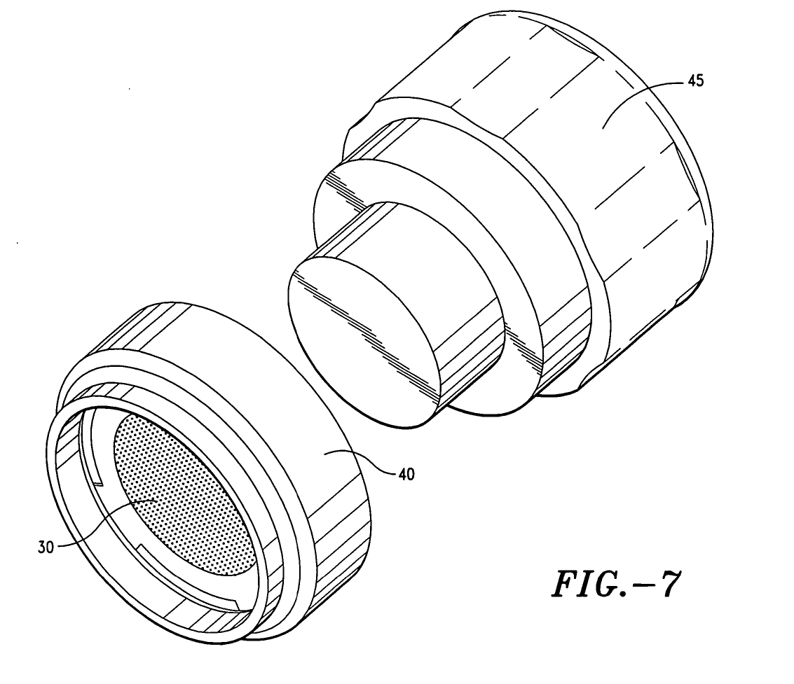

According to the invention, the sheet 36 can be incorporated into a delivery patch, including a backing 40 for the sheet 36, and can additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

In further embodiments of the invention, the biocompatibility of the microprojection member 30 can be improved to minimize or eliminate bleeding and irritation following application to the skin of a subject. Specifically, the microprojections 34 can have a length less than 145 microns, more preferably, in the range of approximately 50-145 microns, and even more preferably, in the range of approximately 70-140 microns. Also, the microprojection member 30 comprises an array preferably having a microprojection density greater than 100 microprojections/cm², and more preferably, in the range of approximately 200-3000 microprojections/cm². Further details regarding microprojection members having improved biocompatibility are found in U.S. Application Ser. No. 60/653,675, filed Feb. 15, 2005, which is hereby incorporated by reference in its entirety.

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymeric material. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. Application No. 60/484,142, which is incorporated by reference herein in its entirety.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

In certain embodiments of the invention, the microprojections 34 are preferably configured to reduce variability in the applied coating 35. Suitable microprojections generally comprise a location having a maximum width transverse to the longitudinal axis that is located at a position in the range of approximately 25% to 75% of the length of the microprojection from the distal tip. Proximal to the location of maximum width, the width of the microprojection tapers to a minimum width. Further details regarding the noted microprojection configurations are found in U.S. Application Ser. No. 60/649,888, filed Jan. 31, 2005, which is incorporated by reference herein in its entirety.

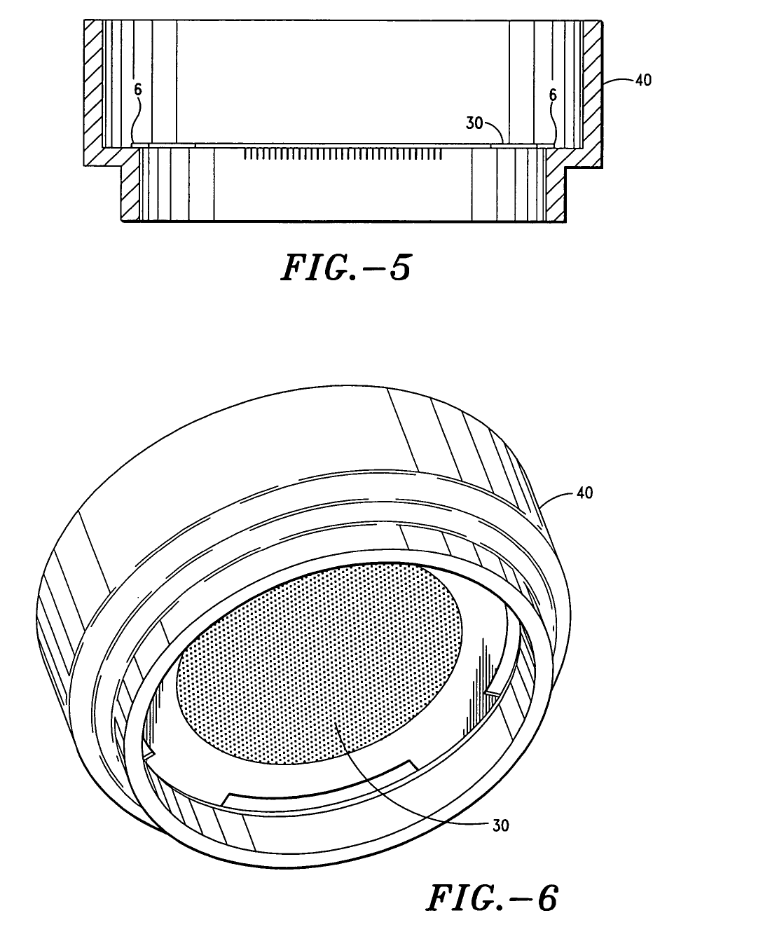

Referring now to FIG. 3, there is shown a microprojection member 30 having microprojections 34 that include a biocompatible coating 35 that includes desmopressin. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

Referring now to FIGS. 5 and 6, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member 30 in the retainer ring 40, the microprojection member 30 is applied to the patient's skin. Preferably, the microprojection member 30 is applied to the patient's skin using an impact applicator 45, such as shown in FIG. 7 and described in Co-Pending U.S. application Ser. No. 09/976,978, which is incorporated by reference herein in its entirety.

As indicated, according to one embodiment of the invention, the coating formulations applied to the microprojection member 30 to form solid biocompatible coatings can comprise aqueous and non-aqueous formulations having desmopressin. According to the invention, desmopressin can be dissolved within a biocompatible carrier or suspended within the carrier.

In the preferred embodiment of the present invention, desmopressin is the active ingredient employed in formulations described herein. It should be understood, however, that the present invention is intended to not only cover desmopressin, but also vasopressin, and all other active fragments, degradation products, salts and simple derivatives and combinations thereof of desmopressin and/or vasopressin. Reference to desmopressin should be understood to also include reference to vasopressin, and all other active fragments, degradation products, salts and simple derivatives and combinations thereof of desmopressin and/or vasopressin.

Examples of suitable desmopressin salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, desmopressin is present in the coating formulation at a concentration in the range of approximately 1-30 wt. %.

More preferably, the amount of desmopressin contained in the biocompatible coating on the microprojection member is in the range of 1-1000 μg, even more preferably, in the range of 10-100 μg.

Preferably, the pH of the coating formulation is below about pH 8. More preferably, the coating formulation has a pH in the range of pH 2-pH 8. Even more preferably, the coating formulation has a pH in the range of approximately pH 3-pH 6.

In certain embodiments of the invention, the viscosity of the coating formulation is enhanced by adding low volatility counterions. In one embodiment, desmopressin has a positive charge at the formulation pH and the viscosity-enhancing counterion comprises an acid having at least two acidic pKas. Suitable acids include, without limitation, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

Another preferred embodiment is directed to a viscosity-enhancing mixture of counterions, wherein desmopressin has a positive charge at the formulation pH and at least one of the counterions comprises an acid having at least two acidic pKas. The other counterion is an acid with one or more pKas. Examples of suitable acids include, without limitation, hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

In the noted embodiments of the invention, the amount of counterion is preferably sufficient to neutralize the charge of desmopressin. In such embodiments, the counterion or the mixture of counterion is preferably sufficient to neutralize the charge present on the agent at the pH of the formulation. In additional embodiments, excess counterion (as the free acid or as a salt) is added to the peptide to control pH and provide adequate buffering capacity.

In one preferred embodiment, the agent comprises desmopressin and the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve a viscosity in the range of about 20-200 cp.

In a preferred embodiment, the viscosity-enhancing counterion comprises an acidic counterion, such as a low volatility weak acid. Preferably, the low volatility weak acid counterion exhibits at least one acidic pKa and a melting point higher than about 50° C. or a boiling point higher than about 170° C. at Pₐₜₘ. Examples of such acids include, without limitation, citric acid, succinic acid, glycolic acid, gluconic acid, glucuronic acid, lactic acid, malic acid, pyruvic acid, tartaric acid, tartronic acid and fumaric acid.

In another embodiment, the counterion comprises a strong acid. Preferably, the strong acid exhibits at least one pKa lower than about 2. Examples of such acids include, without limitation, hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions, wherein at least one of the counterion comprises a strong acid and at least one of the counterions comprises a low volatility weak acid.

Another preferred embodiment is directed to a mixture of counterions, wherein at least one of the counterions comprises a strong acid and at least one of the counterions comprises a weak acid with high volatility. Preferably, the volatile weak acid counterion exhibits at least one pKa higher than about 2 and a melting point lower than about 50° C. or a boiling point lower than about 170° C. at Pₐₜₘ. Examples of such acids include, without limitation, acetic acid, propionic acid, pentanoic acid and the like.

The acidic counterion is preferably present in an amount sufficient to neutralize the positive charge present on desmopressin at the pH of the formulation. In additional embodiments, excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the coating formulation. Preferably the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as Cremophor EL®.

In one embodiment of the invention, the concentration of the surfactant is in the range of approximately 0.01-20 wt. % of the coating formulation. Preferably the surfactant is in the range of approximately 0.05-1 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and inulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at ratio of approximately 0.1-2.0:1 with respect to desmopressin, more preferably, approximately 0.25-1.0:1 with respect to desmopressin.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention is particularly useful to prevent bleeding that can occur following application of the microprojection member or array and to prolong the pharmacokinetics of desmopressin through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Other known formulation adjuvants can also be added to the coating formulations provided they do not adversely affect the necessary solubility and viscosity characteristics of the coating formulation and the physical integrity of the dried coating.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In accordance with one embodiment of the invention, the method for delivering desmopressin contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

Preferably, the amount of desmopressin contained in the biocompatible coating (i.e., dose) is in the range of approximately 1 μg-1000 μg, more preferably, in the range of approximately 10-200 μg per dosage unit. Even more preferably, the amount of desmopressin contained in the biocompatible coating is in the range of approximately 10-100 μg per dosage unit.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

In several embodiments of the present invention, a method is disclosed for transdermally delivery desmopressin to a subject. Subject or patient, as used in this application means a human being. As previously mentioned, in one embodiment of the present invention, subject means a child. In another embodiment of the present invention, subject means a geriatric patient, and a method is disclosed for treating enuresis, diabetes insidipus, and other similar types of diseases known to be treated by desmopressin, utilizing a delivery system having a microprojection member (or system) coated with desmopressin. While it is known that desmopressin reduces night urine volume in geriatric patients: implication for treatment of the nocturnal incontinence. Seiler W O, Stahelin H B, Hefti U; Clin Investig (July 1992) 70(7):619, desmopressin is not an approved indication for the treatment of enuresis in geriatrics. Altghough the potential use of desmopressin for enuresis in geriatrics has been described, the use of desmopresin for enuresis in geriatric patients is not an approved clinical indication, possibly because the present routes of delivery are cumbersone and/or inefficient. Indeed administration by injection is poorly suited for routine use and intranasal and oral administration result in low and variable bioavailability. It is the belief of the inventors that precise delivery of desmopressin is essential for acceptable, tolerable, and successful treatment in geriatric enuretic patients. A delivery system having a microprojection member (or system) coated with desmopressin will target geriatrics with primary nocturnal enuresis and it is believed to be a more acceptable route of administration with potentially good bioavailability relative to other modes of drug delivery.

It would be advantageous if geriatrics could be treated with a delivery system having a microprojection member (or system) coated with desmopressin. The prevalence of incontinence increases with age and affects more women than men, until after age 80, when men are equally affected. Of persons 65 years and older, 15% to 30% in the community and up to 50% in long-term care are incontinent. Urinary incontinence (UI) can cause morbidity, from cellulitis, pressure ulcers, urinary tract infections, falls with fractures, sleep deprivation, social withdrawal, depression, and sexual dysfunction. UI is not associated with increased mortality. Its impact on quality of life is more a consequence of embarrassment and activity interference than of an effect on activity performance. Caregiver burden is higher with incontinent older persons and contributes to decisions to institutionalize. Estimated annual UI-related costs total more than $16 billion (Catherine E. DuBeau, Clinical Geriatrics—ISSN: 1070-1389—Volume 09—Issue 05—May 2001)

It is the belief of the inventors that there is an unmet medical need for treatment of enuresis in the geriatric target population. Indeed an article in Clinical Geriatrics states “The prevalence of incontinence increases with age and affects more women than men, until after age 80, when men are equally affected. Of persons 65 years and older, 15% to 30% in the community and up to 50% in long-term care are incontinent. Urinary incontinence (UI) can cause morbidity, from cellulitis, pressure ulcers, urinary tract infections, falls with fractures, sleep deprivation, social withdrawal, depression, and sexual dysfunction. UI is not associated with increased mortality. Its impact on quality of life is more a consequence of embarrassment and activity interference than of an effect on activity performance. Caregiver burden is higher with incontinent older persons and contributes to decisions to institutionalize. Estimated annual UI-related costs total more than $16 billion (Catherine E. DuBeau , Clinical Geriatrics—ISSN: 1070-1389—Volume 09—Issue 05—May 2001)”.

A delivery system having a microprojection member (or system) coated with desmopressin will target geriatrics with primary nocturnal enuresis and it is believed to be a more acceptable route of administration with potentially good bioavailability relative to other modes of drug delivery.

### EXAMPLES

The following examples are given to enable those skilled in the art to more clearly understand and practice the present invention. They should not be considered as limiting the scope of the invention, but merely as being illustrated as representative thereof.

### Example 1

A study was performed to explore the feasibility of delivering desmopressin transdermally through the skin by means of microneedle technology, which uses a microneedle array to overcome the skin barrier. The hairless guinea pig (HGP) was chosen as the animal model for the preclinical study because its skin anatomy is more similar to human skin than that of rodent. In addition, HGP is known to be a good experimental model for drug transport, skin irritation, and wound healing. The patch technology incorporated an array of titanium microneedles that created superficial pathways through the skin barrier for transport of therapeutics and vaccines. The tips of microneedles in 2-cm² arrays were covered with a solid coating of various amounts of desmopressin and applied to the skin of hairless guinea pigs for 5 or 15 min. Pharmacologically relevant amounts of desmopressin were delivered after 5 minutes. Bioavailability was as high as 85% and showed acceptable variability (30%). Immunoreactive serum desmopressin reached peak levels after a Tₘₐₓ of 60 min. Elimination kinetics for serum desmopressin were similar after transdermal and IV delivery, suggesting absence of a skin depot. Only 10% of the desmopressin dose loaded onto the microneedle array was found on the skin surface after application. Additionally, the patches were well tolerated. These results suggest that transdermal delivery of desmopressin by the microneedle array is a safe and efficient alternative to currently available routes of administration.

Materials and Methods

Animals

Outbred male and female euthymic HGPs were obtained from Biological Research Labs (Switzerland, strain ibm:GOHI-hr) and Charles River Labs (Michigan, strain IAF:HA-HO-hr). The animals, which weighed 500 to 1000 g, were quarantined, individually housed, and maintained in a facility accredited by the Association for Assessment and Accreditation of Laboratory Animal Care. All research protocols adhered to the Principles of Laboratory Animal Care (NIH publication #85-23, revised 1985).

Microneedle Array

Microneedle arrays were produced by photo-chemical etching and forming using a controlled manufacturing process. The finished microneedle array was a titanium screen with defined microneedle density and length. Microneedles were arranged in a hexagonal close-packed pattern with 321 microneedles/cm² over an area of 2 cm². Each microneedle was arrowhead-shaped with a length of 200 μm, a maximal width of 170 μm, and a thickness of 35 μm.

Desmopressin Coating

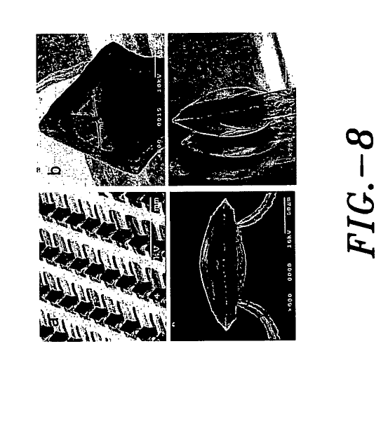

Desmopressin acetate was obtained from Diosynth B. V., Netherlands. Microneedle arrays were coated with drug by partial immersion in aqueous formulations containing 40 wt % desmopressin and 0.2 wt % polysorbate 20. For urinary excretion studies, ³H-desmopressin (3,4,5-phenylalanyl-³H-desmopressin, 68.5 Ci/mmol, Perkin Elmer, Boston, Mass.) was added as a tracer to unlabeled desmopressin to a final specific activity of 2000-4000 dpm/μg. Coating was performed in ambient conditions (22° C., 45% RH) using an apparatus that limited application of the drug to the tip of the microneedles. FIG. 8 shows a desmopressin-coated array. All finished arrays were visually evaluated for coating homogeneity by light stereo-microscopy, and obviously contaminated arrays (ie, those arrays on which the coating was found to extend to the base of the microprojections) were discarded. Adhesion of the coating to the titanium substrate was probed manually with a 30-gauge needle under a light stereo-microscope. Coating depth and thickness were evaluated on selected arrays by scanning electron microscopy.

Desmopressin Content and Purity

To validate the amount of desmopressin present on the array, representative arrays were soaked in 1 mL water for 10 min, and the absorbance of the aqueous extract was measured at 275 nm. In cases where ³H-labeled desmopressin had been added to the coating solution, arrays were extracted in 3 mL water, and the radioactive concentration of the extract was measured by scintillation counting.

For evaluation of desmopressin stability following coating, coated arrays were stored at 25° C. in a nitrogen atmosphere for 1, 2, 3, and 6 months. At each time-point, ten arrays were separately extracted in 1 mL water for 10 min. Desmopressin content and purity in the aqueous extract were analyzed by RP-HPLC on a stainless steel column packed with octadecylsilyl silica gel (3.5 μm), using a mixture of 60 volumes of mobile phase A (0.067 M phosphate buffer pH 7.0) and 40 volumes of mobile phase B (equal volumes of mobile phase A and of acetonitrile) at a flow rate of 0.4 mL/min for isocratic elution and UV spectroscopy at 220 nm for detection.

Patch Assembly

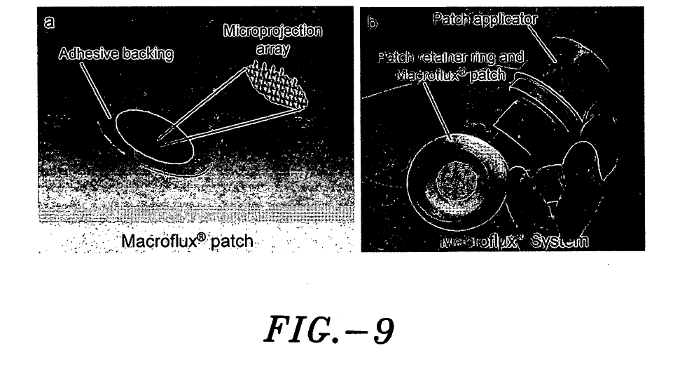

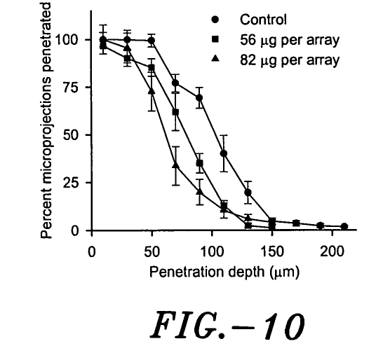

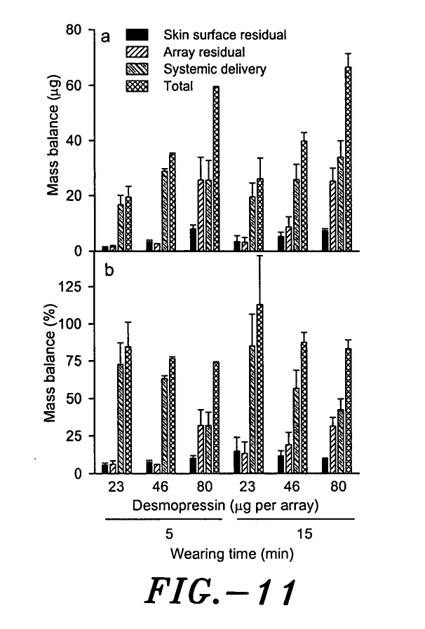

Following coating, the microneedle array was affixed to an adhesive patch composed of a low-density polyethylene backing with an acrylate adhesive. The final systems had a total patch area of 5.3 cm², including the 2-cm² microneedle array. The patch was loaded onto a disposable retainer ring and stored under a nitrogen atmosphere at 4° C. for up to 6 months. FIG. 9 shows the different elements of the microneedle array delivery system, including the impact applicator.

Patch Application

HGPs were anesthetized using a gas delivery system (isoflurane 3-3.5%, 2-2.5 L O₂/minute), and treatment sites on the lateral skin areas of the thorax were cleaned with 70% isopropanol wipes and allowed to dry. The retainer ring containing the patch was loaded onto an impact applicator, and the patch was applied to the prepared skin site with an impact energy of 0.26 J, delivered in less than 10 ms. The patch was worn for either 5 or 15 min.

Penetration Assessment

The depth of microneedle penetration was evaluated as previously described using an India ink skin distribution technique. Briefly, the patch was removed immediately after application to the HGP, and the skin site was dyed with a cotton swab imbibed with India ink. A series of skin biopsies of the application site was sectioned parallel to the skin surface, and the number of stained pathways in each slice was recorded. The percentage of microneedles penetrating into the skin was plotted as a function of depth, and the depth at which 50% of the microneedles penetrated (D₅₀) was extrapolated.

Desmopressin Delivery Evaluation

To determine the amount of desmopressin transferred to the skin surface, skin sites were cleaned with two cotton swabs imbibed with 1% SDS and dried with a third cotton swab immediately following patch removal. Used cotton swabs were soaked overnight in 3 mL water to elute the desmopressin. To quantify residual amounts of desmopressin remaining on the arrays, these were separated from the adhesive backing by immersion in liquid nitrogen vapor and soaked in 3 mL water to elute the desmopressin. Desmopressin concentration in aqueous extracts from the cotton swabs and the arrays was determined by scintillation counting.

For urinary excretion studies, the HGPs were housed individually in metabolic cages, and urine was collected for two days following patch removal. ³H-desmopressin content in urine was determined by scintillation counting and used to estimate the amount of desmopressin that had been delivered transdermally, based on the urinary excretion rate (71%) measured after intravenous injection of 20 μg desmopressin. Efficiency of delivery was calculated as the percentage delivered of the total dose coated onto the array. For mass balance calculations, drug residuals left on the skin and on the array were also taken into account.

For pharmacokinetic studies, the HGPs were housed individually in standard cages for the duration of the study. The patch application time was 15 min. Following patch removal, blood samples were taken from the vena cava at two randomly assigned time points in each anesthetized guinea pig (a total of 24 HGPs were used for this study). Serum desmopressin levels were determined by a radioimmunoassay (MDS Pharma Services) with a limit of detection of 40 pg/mL. The amount of desmopressin delivered from the microneedle array was estimated by comparing the AUC for the serum desmopressin concentration after transdermal delivery to the AUC for the serum desmopressin concentration after IV injection of 11 μg desmopressin in a separate group of animals (20 HGPs).

Skin Response

Following system removal, skin sites were visually assessed for signs of erythema, edema, bleeding, and infection until the sites returned to normal.

Statistical Analysis

Results are presented as the mean of three to four determinations with its associated standard error of the mean (SEM). Statistical analysis was performed by two-way analysis of variance and t-test. A probability value of p<0.05 was considered statistically significant.

Results

Desmopressin Stability and Coating Morphology

Stability studies demonstrated that greater than 98% of the desmopressin coated onto microneedle arrays remained intact for at least 6 months under the storage conditions described. Scanning electron microscopy revealed good uniformity of coating from microneedle to microneedle (FIG. 8a), with coating limited to the first 100 μm of the microneedle tip. After two days of storage in a nitrogen atmosphere, the solid coating presented a smooth surface with some cracking (FIG. 8b) and adhered tightly to the microneedles. On each individual microneedle, the coating was found to present a distinct distribution pattern. Most of the solid coating appeared to be located in spherical caps centered to the geometic centers of the coated areas of the two faces of the microneedle (FIGS. 8c and 8d). The maximum measured thickness of the coating was about 15 μm on each side of the microneedle for an array loaded with 80 μg desmopressin.

Skin Penetration

Skin penetration studies showed that almost 100% of uncoated microneedles penetrated the first 50 μm of the skin. The D₅₀ was about 100 μm, and only 5% of the microneedles penetrated to a depth of 150 μm. Coating with desmopressin decreased the penetration depth of the microneedles significantly. Specifically, coating with 56 μg desmopressin reduced the D₅₀ to about 75 μm, and coating with 82 μg desmopressin reduced the D₅₀ to about 60 μm. Under all coating conditions tested, however, more than 90% of the microneedles penetrated the first 30 μm of the skin (FIG. 10) and thus passed through the stratum corneum, which is approximately 20-30 μm thick.

Desmopressin Delivery

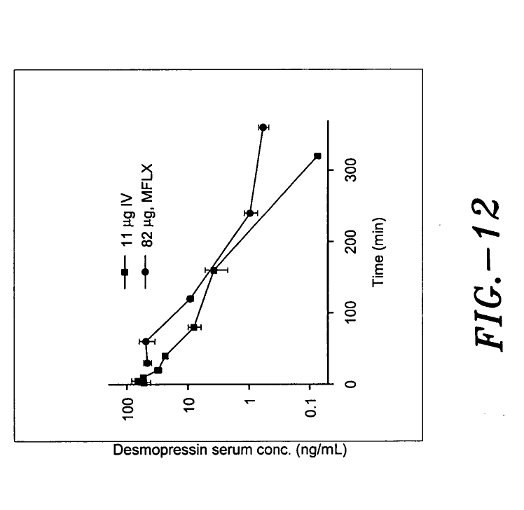

The amount of desmopressin delivered from microneedle arrays coated with desmopressin was determined for different desmopressin loadings and different patch wearing times. The total absolute amount of desmopressin recovered was calculated from the amount of desmopressin delivered systemically, the amount deposited on the skin surface, and the amount remaining on the array following patch removal (FIG. 11a) and presented as a percentage of the loading dose (FIG. 11b). Absolute desmopressin delivery from coated arrays did not vary significantly with wearing time or loading dose. Average delivery ranged from 17 to 34 μg, with an overall average of 25 μg. Conversely, when the delivery results were expressed as a percentage of the total dose coated onto the array, an increased loading dose resulted in a significant decrease in drug delivery efficiency or bioavailability. Drug delivery efficiency varied from an average of 79% at the lowest loading dose to an average of 37% at the highest loading dose (5-min and 15-min wearing times combined). A maximum drug delivery efficiency of 85% was achieved following 15-min wearing at the lowest loading. This decrease in drug delivery efficiency with increased loading dose was paralleled by an increase in residual drug found on the used microneedle array from an average of 10% at the lowest loading dose to an average of 32% at the highest loading dose. While the absolute amount of desmopressin recovered from the skin surface increased from 1.3 μg following application of an array loaded with 23 μg desmopressin to 8 μg following application of an array loaded with 80 μg desmopressin (5-min wear time), it remained at about 10% of the total loading dose under all conditions tested. Total desmopressin recovery ranged from an average of 74% to 113%.

Following application of arrays coated with 82 μg desmopressin, serum concentrations of desmopressin were evaluated using an immunoassay, and pharmacokinetic parameters were estimated. A Cₘₐₓ of 49 ng/mL was reached at a Tₘₐₓ of 60 min, and the AUC of the serum desmopressin—time curve was calculated as 76 (ng×h)/mL. By comparing this AUC to an AUC of 50.5 (ng×h)/mL after IV administration of 11 μg desmopressin, the desmopressin dose delivered by the microneedle array was extrapolated to be 17.5±3.8 μg. FIG. 12 shows the desmopressin serum concentrations following IV and microneedle array administration. Elimination kinetics for serum desmopressin were similar after delivery by microneedle array or IV.

Tolerability

The microneedle patches were well tolerated by the HGPs. Following removal of the systems, only mild erythema was observed that typically resolved within 24 h. In addition, no signs of edema, bleeding, or infection, were observed in any of the animals

Discussion

Transdermal delivery represents a desirable route of administration for desmopressin, a synthetic peptide hormone with low oral and nasal bioavailability, because it offers a less painful and invasive way of administration than injection. However, delivery of desmopressin through intact skin is known to be negligible both in humans and, in studies conducted by the present authors, in the HGP animal model (unpublished observation). Here, the feasibility of using a microneedle array coated with desmopressin to promote transdermal desmopressin delivery is being evaluated. Needle coating technology has been used for many years to introduce antigens into the skin for diagnostic purposes, but no published reports exist of controlled systemic delivery of drugs from drug-coated microneedles. For the studies described here, a custom-made coating apparatus was used to deposit drug on the tip of the microneedles. This technique is still at an early stage, and reasonable reproducibility was achieved only through manual elimination of contaminated arrays. However, progress has been made in the coating technology since these studies were performed, and coating can now be performed with greater reproducibility and a lower rate of contamination.

On the microneedle arrays used here, the coating appeared to be located in spherical caps centered to the geometic centers of the coated areas of the two faces of the microneedle. This is a somewhat counterintuitive result given the fact that arrays are coated and dried with the tips of the microneedles facing down. The coating pattern suggests that surface tension is the predominant force determining distribution of coating at the small scale involved. Overall, the coating showed good aerodynamics and good adhesion, consistent with the minimal effect of coating on penetration of the microneedles through the stratum corneum barrier and the minimal loss of drug from the microneedles on the skin surface during penetration through the skin.

For drug coated onto the tips of microneedles to be delivered reproducibly, the microneedles have to penetrate uniformly beyond the stratum corneum barrier. The impact applicator used here exerted a predetermined force for application of the system, leading to reproducible penetration depth of the microneedles. This was shown by the penetration-depth profile of the microneedles into the skin, visualized with India ink staining. In addition, the impact applicator is easy to use, reusable, and should facilitate acceptability of this delivery system in a clinical setting.

Desmopressin residuals on the skin following patch removal were found to be only a fraction (10%) of the total dose coated. This finding demonstrates that the drug is not dislodged from the microneedles during the penetration process, but consistently delivered into the skin. Also, although the drug is administered into the uppermost layers of the skin, it is not extractable by extensive cleaning of the skin surface as demonstrated in the studies described here. The minimal skin contamination seen with this system is beneficial from a safety and environmental standpoint.

Increase in drug loading of the microneedle arrays resulted in only a slight increase in the absolute amount of drug delivered into the skin, while a significant decrease in drug delivery efficiency was observed. This result is not completely understood. It is unlikely that this result is due to a reduction in penetration depth with increased loading, since the penetration-depth experiments demonstrated that more than 90% of the microneedles penetrated the skin beyond the stratum corneum barrier using a desmopressin loading of up to 82 ug. Additional experiments conducted with similar high desmopressin loadings and longer wearing times of up to 1 h did not demonstrate appreciable increase in desmopressin delivery (data not shown). Therefore, a likely explanation for the reduced bioavailability with increased drug loading could be the limited availability of interstitial fluids for desmopressin dissolution. This hypothesis is consistent with the increase in drug residual on the arrays found with increase drug loading, indicating that dissolution of the coating is the limiting factor for the observed reduced bioavailability. At low loading dose, however, drug delivery is optimal while drug residual on the array is minimal, indication that drug dissolution is not a limiting factor.

Drug utilization observed with the coated microneedle array can be as high as 85% with low desmopressin loading following 15 min wearing. A delivery of 20 ug desmopressin was achieved with this condition, which is within the target clinical dose of 1-20 ug. Reduced drug loading would likely allow adjustment of the dose delivered to a lower clinical target with a similarly high delivery efficiency. Unfortunately, in practice, such low loading would have been difficult to monitor for coating homogeneity and contamination using light stereo-microscopy.

Reproducibility of delivery was found to be acceptable. Under all conditions tested, the SEM was less than 30% of the mean. This variability is compatible with clinical administration of desmopressin, which has a large therapeutic window.

No differences in delivery were observed between 5 and 15 min wearing time. These data indicate that a wearing time as short as 5 min is sufficient for transdermal desmopressin delivery and that longer wearing times do not result in additional delivery. Therefore, patch wear does not have to be precisely timed, making the system potentially safe, patient compliant, and user-friendly.

The pharmacokinetic profile of desmopressin following administration by coated microneedle array also suggested fast delivery of the drug. The absorption and distribution phase was complete at 60 min after patch application. Following this phase, desmopressin elimination paralleled that observed after IV administration, suggesting the absence of a significant skin depot of the drug. This minimal skin depot renders the pharmacokinetics of transdermally delivered desmopressin more predictable and thus adds to the safety of this route of administration. Results obtained with the pharmacokinetic model were comparable to those obtained with the urinary excretion model. Serum levels were determined with a specific immunoassay, which measured immunoreactivity rather than bioactivity of desmopressin. However, considering the small size of the peptide, it is likely that the immunoassay detected bioactive desmopressin. Following these preclinical studies, a clinical study, performed with microneedle arrays coated with desmopressin, validated the results presented here.

Conclusions

In these studies, a target dose of 20 μg desmopressin was delivered to hairless guinea pigs from a 2-cm² microneedle array within 15 minutes. Transdermal delivery was efficient and showed acceptable variability, and the dose and delivery efficiency could be adjusted by varying the loading dose on the microneedle array. Desmopressin residual on the skin surface was minimal and pharmacokinetic data suggested absence of skin depot. Additionally, the microneedle patches were well tolerated in all animals. These results suggest that transdermal delivery of desmopressin by a microneedle array is a safe and efficient alternative to currently available routes of administration.

### Example 2

15% w/w desmopressin, 30% w/w sucrose, 0.2% polysorbate 20 solution was coated on 2 cm² microneedle arrays. The tips of microneedles in 2-cm² arrays were covered with a solid coating of 25 mcg of desmopressin. The pharmacokinetic/pharmacodynamic (PK/PD) and topical safety profiles of desmopressin administered by the titanium microneedle array patch system (“MFLX”) of Example 1 were evaluated.

Methods: This was a 2-part study in healthy human volunteers (18-45 years). The mean (SD) demographics were age (years): 26.2 (6.5); height (cm): 174.3 (10.7); weight (kg): 69.4 (11.9); sex: male=9, female=15; and ethnic origin: Caucasian=22, black=1, other=1. In Part I, 8 subjects received 30 ug desmopressin by intravenous (IV) infusion (1 ug/min) and a MFLX 25-ug desmopressin patch, sequentially. In Part II, 16 subjects received 15 ug desmopressin by IV infusion (iug/min) and a MFLX 25-ug desmopressin patch in a randomized, crossover fashion. The MFLX patch was worn on the upper outer arm for 15 minutes in both Parts I and II. All treatments were administered 30 minutes after a subcutaneous injection of low-molecular weight heparin to counteract the increase in clotting factors in the healthy volunteer population. Blood samples were taken over 32 hours post treatment to evaluate serum desmopressin concentrations (validated by radioimmunoassay) and their pharmacodynamic effect on plasma coagulation Factor VIII levels (one-stage clotting assay). Topical effects were also examined. Pain upon patch application was scored and compared to subcutaneous saline injection with a 27-gauge needle. Pain, pressure, and sensation assessment were measured using numerical scale and questionnaire after the following: subcutaneous 0.2 mL saline (used as control), MFLX application, and MFLX removal. Bleeding assessment (visual and photographic) were measured 2 min post-system removal. Erythema assessment were measured up to 32 hours post-dose initiation. Routine safely assessments were also included, including the following: solicitation of Adverse Events (AEs), measurement of vital signs, measurement of serum sodium concentration and platelets, and measurement of urine output, specific gravity and osmolality.

Data analysis included a review of PK parameters (Cₘₐₓ, Tₘₐₓ, elimination rate constant, apparent half-life, and AUC_{inf}) and were estimated using standard methodology. The amount of desmopressin absorbed from MFLX was estimated as follows: [AUC_{inf}(MFLX)/AUC_{inf}(IV)]×Dose (IV). The intra-individual (sigma) and inter-individual (CV %) variabilities were estimated using additive+proportional and log normal distributions, respectively. Model performance between hierarchical models was assessed both numerically (Objective Function, OBJ) and graphically (comparison of the predicted and observed data). A reduction of³ 6.7 units in OBJ per additional structural model parameter was considered significant at p<0.01 on the x² distribution.

Results: Following MFLX application, desmopressin was rapidly absorbed with peak concentrations by 25 minutes. The mean amount absorbed (5.5 ug) is within the dose range (2-8 ug) for its antidiuretic effects. The mean half-life value following MFLX (2.8 hours) was similar to IV (3.1-3.8 hours). The mean desmopressin Cmax values were 1903, 1321, and 269 pg/mL following IV-30 ug, IV-15 ug, and MFLX treatments, respectively. Increases in Factor VIII values (321, 280, and 166% of baseline value) indicated that the absorbed drug was pharmacologically active. Topical effects for MFLX were none to mild for most subjects. A larger number of subjects had no pain (62.5%) upon MFLX application vs saline injection (24%).

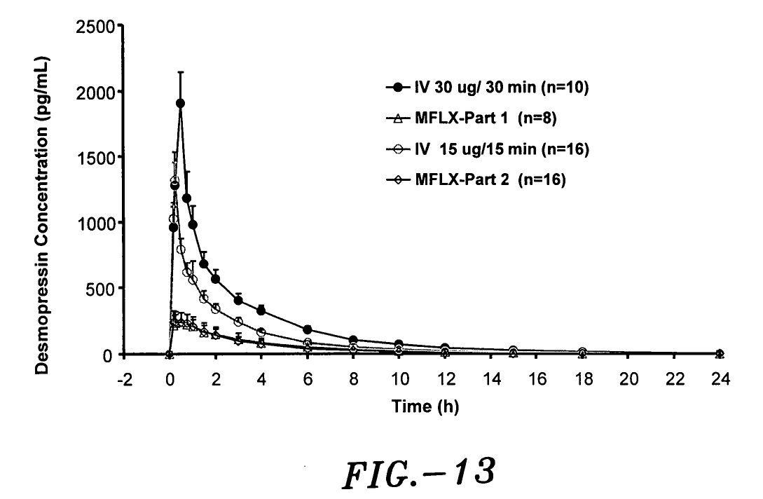

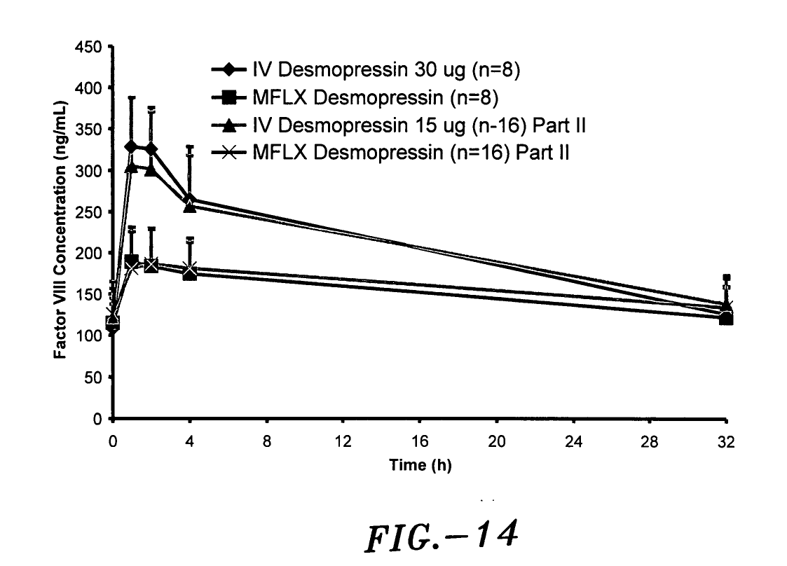

The mean desmopressin concentrations-time profiles are presented in FIG. 13 and PK parameters are presented in Table 1.

TABLE 1

| | Treatments | | |
|---|---|---|---|
| Parameter | IV - 30 μg | IV - 15 μg | MFLX |
| N | 8 | 16 | 24 |
| Cₘₐₓ (pg/mL) | 1903 ± 267 | 1321 ± 134 | 269 ± 79 |
| | (14) | (10) | (29) |
| Tₘₐₓ (h) | 0.50 | 0.26 | 0.43 ± 0.23 |
| | | | (53) |
| t_{1/2} (h) | 3.1 ± 0.38 | 3.8 ± 1.25 | 2.8 ± 0.52 |
| | (12) | (33) | (19) |
| AUC_{inf} (pg · h/mL) | 4027 ± 355 | 2338 ± 204 | 902 ± 294 |
| | (9) | (9) | (33) |
| Amt Absorbed (μg) | Reference | Reference | 5.5 ± 1.8 |
| | | | (33) |

| | |
|---|---|
| Nominal Amt Coated (μg) | 25 |
| % of Nominal Amt Absorbed | 22 |

Following MFLX application, desmopression was rapid absorbed and mean peak plasma concentration of 269 pg/mL was noted at 25 min. Approximately 5.5 mg desmopression was absorbed from the MFLX system, which is within the dose range for antidiuretic effect (2-8 mg). Bioavailability of MFLX desmopressin was approximately 22%. Mean terminal half-life was similar between IV and MFLX desmopressin.

FIG. 14 presents the mean (SD) plasma factor VIII concentration-time profiles following IV and MFLX treatments. FIG. 14 demonstrate increases in Factor VIII values indicating that the absorbed desmopressin was pharmacologically active.

Tables 2-4 present the application site reaction assessments for MFLX patches.

TABLE 2
Pain Assessment Results for MFLX Desmopressin Treatment

| | | Saline | MFL | MFL |
|---|---|---|---|---|
| | | Injection^{b} | Application | Removal |
| | | No. (%) | No. (%) | No. (%) |
| Assessment | Score^{a} | N = 25 | N = 24 | N = 24 |
| Pain | 0 | 24.0 | 62.5 | 87.5 |
| | 1 | 16.0 | 25.0 | 12.5 |
| | 2 | 28.0 | 4.2 | 0.0 |
| | 3 | 16.0 | 4.2 | 0.0 |
| | 4 | 4.0 | 4.2 | 0.0 |
| | 5 | 4.0 | 0.0 | 0.0 |
| | 6 | 8.0 | 0.0 | 0.0 |

TABLE 3
Pressure/Sensation/Bleeding Assessment
Results for MFLX Desmopressin Treatment

| | | MFL | | |
|---|---|---|---|---|
| | | Application | MFL Removal | |
| | | No. (%) | No. (%) | |
| Assessment | Score | N = 24 | N = 24 | |
| Pressure | None | 20.8 | 87.5 | |
| | Mild | 58.3 | 12.5 | |
| | Moderate | 20.8 | 0.0 | |
| Sensation | None | 66.7 | 91.7 | |
| | Sharp | 4.2 | 0.0 | |
| | Pricking | 8.3 | 4.2 | |
| | Dull | 0.0 | 4.2 | |
| | Tingling | 20.8 | 0.0 | |
| | Smarting | 4.2 | 0.0 | |
| Bleeding | None | Not Applicable | 45.8 | |
| Area | <25% | Not Applicable | 54.2 | |
| No. of | | Not Applicable | 1-8 | >20 |
| blood spots | | | (n = 12) | (n = 1) |

TABLE 4
Erythema Assessment Results for MFLX Desmopressin Treatment

| | 0 HR | 12 HR | 24 HR | 32 HR |
|---|---|---|---|---|
| | No. (%) | No. (%) | No. (%) | No. (%) |
| Score - Time Post Removal | N = 24 | N = 23 | N = 23 | N = 24 |
| None | 12.5 | 13.0 | 30.4 | 41.7 |
| Noticeable redness | 62.5 | 65.2 | 60.9 | 54.2 |
| Well- defined redness | 25.0 | 21.7 | 8.7 | 4.2 |
| Beet redness | 0.0 | 0.0 | 0.0 | 0.0 |

The following were evident from the data presented in Tables 2-4: majority of subjects did not experience any pain at MFLX application (62.5%) or removal (87.5%); majority of subjects felt only a mild pressure at MFLX application (58.3%), and no pressure at MFLX removal (87.5%); majority of subjects did not experience any sensation at MFLX application (66.7%) or removal (91.7%); at MFLX removal, 45.8% of subjects had no bleeding and 54.2% had <25% application area bleeding; and majority of subjects had mild erythema (noticeable redness) at MFLX removal (62.5%) that lasted up to 32 hours post-removal (54.2%).

Conclusion:

With regard to PK, serum desmopressin concentration-time profiles following IV administration were consistent with the duration of IV infusion with peak concentrations at 30 minutes and 15 minutes in Parts 1 and 2, respectively and best described by a 3-compartmental model. Serum desmopressin concentration-time profiles after MFLX administration had the following characteristics: they indicated fast drug absorption with peak concentration at 25 minutes post patch application; and they were best described by a 3-compartmental model with first-order absorption, and estimated Ka of 2.56 h-1 also suggested rapid absorption. The mean terminal half-life was similar between IV and MFLX treatments, indicating minimal skin depot. Approximately 22% of the drug contained in the MFLX patch was absorbed. The amount absorbed (5.5 mg) was within the dose range for the antidiuretic effect.

With regard to PD, results of factor VIII measurements indicated that absorbed desmopressin was pharmacologically active.

With regard to safety, application/removal of MFLX patches did not result in significant discomfort, skin topical effects were mostly mild, and MFLX desmopressin was well tolerated

Overall, the MFLX 25-ug desmopressin patch demonstrated rapid delivery and the amount absorbed was in the therapeutically active dose range. Topical effects and pain reception were in the none to mild category.

As will be appreciated by one having ordinary skill in the art, the present invention provides numerous advantages. For example, a microprojection based apparatus and method has the advantage of transdermal delivery of a desmopressin-based agent exhibiting a desmopressin-based agent pharmacokinetic profile similar to that observed following intravenous administration. While a subcutaneous leg was not presented in the examples, the literature indicates that the elimination kinetics following subcutaneous and intravenous adminstration are similar. Therefore, a microprojection based apparatus and method should have the advantage of transdermal delivery of a desmopressin—based agent exhibiting a desmopressin-based agent pharmacokinetic profile similar to that observed following subcutaneous administration (see for example: Eur J Clin Pharmacol. 1988; 35(3):281-5; Thromb Haemost. Dec. 18, 1987; 58(4):1037-9; and Thromb Haemost. Feb. 28, 1986; 55(1):108-11). Another advantage is transdermal delivery of a desmopressin-based agent with rapid on-set of biological action. Yet another advantage is transdermal delivery of a desmopressin-based agent with sustained biological action for a period of up to 10 hours. Further, transdermal delivery from a microprojection array coated with a 10-100 μg dose of desmopressin results in a plasma Cₘₐₓ of at least 50 pg/mL after one application.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A delivery system for transdermally delivering desmopressin to a patient, comprising:
a microprojection member having a plurality of microprojections that are adapted to pierce the stratum corneum of the patient; and
a biocompatible coating disposed on said microprojection member, said coating being formed from a coating formulation having desmopressin disposed therein.

2. The delivery system of claim 1, wherein said coating is disposed on at least one of said plurality of microprojections.

3. The delivery system of claim 1, wherein said coating formulation comprises an aqueous formulation.

4. The delivery system of claim 1, wherein said coating formulation comprises a non-aqueous formulation.

5. The delivery system of claim 1, wherein said desmopressin is selected from the group consisting of desmopressin, arginine vasopressin, vasopressin analogs and combinations thereof.

6. The delivery system of claim 5, wherein said desmopressin is selected from the group consisting of active fragments, degradation products, salts, simple derivatives and combinations thereof of desmopressin, arginine vasopressin and vasopressin analogs.

7. The delivery system of claim 6, wherein said desmopressin is desmopressin.

8. The delivery system of claim 1, wherein desmopressin comprises in the range of approximately 1-30 wt. % of said coating formulation.

9. The delivery system of claim 1, wherein desmopressin comprises in the range of 1 μg-2000 μg of said biocompatible coating.

10. The delivery system of claim 1, wherein the pH of said coating formulation is below approximately pH 8.

11. The delivery system of claim 1, wherein said coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarbally acid, malonic acid, adipic acid, citraconic acid, glutaratic acid, itaconic acid, mesaconic acid, citramalic acid, dimethylopropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

12. The delivery system of claim 1, wherein said coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, alkoxylated alcohols and mixtures thereof.

13. The delivery device of claim 1, wherein said coating formulation includes at least one polymeric material having amphiphilic properties.

14. The delivery system of claim 1, wherein said coating formulation includes a hydrophilic polymer selected from the following group consisting of hydroxyethyl starch, dextran, poly(vinyl alcohol), poly(ethylene oxide), poly(2-hydroxyethyl-methacrylate), poly(n-vinyl pyrolidone), polyethylene glycol and mixtures thereof.

15. The delivery system of claim 1, wherein said coating formulation includes a biocompatible carrier selected from the group consisting of human albumin, bioengineered human albumin, polyglutamic acid, polyaspartic acid, polyhistidine, pentosan polysulfate, polyamino acids, sucrose, trehalose, melezitose, raffinose, stachyose, mannitol and like sugar alcohols.

16. The delivery system of claim 1, wherein said coating formulation includes a stabilizing agent selected from the group consisting of a non-reducing sugar, a polysaccharide and a reducing sugar.

17. The delivery system of claim 1, wherein said coating formulation includes at least one vasoconstrictor selected from the group consisting of amidephrine, cafaminol, cyclopentaimine, deoxyepinephrine, epinephrine, felypressin, indanzoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline, and mixtures thereof.

18. The delivery system of claim 1, wherein said coating formulation includes at least one pathway patency modulator selected from the group consisting of osmotic agents, zwitterionic compounds, anti-inflammatory agents and anticoagulants.

19. The delivery system of claim 1, wherein said coating formulation has a viscosity in the range of approximately 3-500 centipose.

20. The delivery system of claim 1, wherein the thickness of said biocompatible coating is less than approximately 25 microns.

21. A method of transdermally delivering desmopressin to a patient, comprising the steps of:
providing a microprojection member having a plurality of microprojections, said microprojection member having a coating disposed thereon, said coating including desmopressin;
applying said microprojection member to a skin site of said patient, whereby said plurality of microprojections pierce the stratum corneum and deliver said desmopressin to said patient; and
removing said microprojection member from said skin site.

22. The method of claim 21, wherein said microprojection member remains applied to said skin site for a period of time in the range of 5 sec. to 24 hrs.

23. The method of claim 21, wherein said desmopressin is selected from the group consisting of desmopressin, arginine vasopressin, vasopressin analogs and combinations thereof.

24. The method of claim 21, wherein said desmopressin is selected from the group consisting of active fragments, degradation products, salts, simple derivatives and combinations thereof of desmopressin, arginine vasopressin and vasopressin analogs.

25. The method of claim 21, wherein said desmopressin is desmopressin.

26. The method of claim 21, wherein said desmopressin comprises in the range of approximately 1 μg-2000 μg of said biocompatible coating.

27. The method of claim 21, wherein said delivery of said desmopressin exhibits improved pharmacokinetics compared to the pharmacokinetic characteristics of intravenous or subcutaneous delivery.

28. The delivery system of claim 1 wherein the patient is a child.

29. The method of claim 21, wherein the patient is a child.

30. The delivery system of claim 1 wherein the patient is an adult.

31. The method of claim 21, wherein the patient is an adult.

32. The delivery system of claim 1 wherein the patient is a geriatric patient.

33. The method of claim 21, wherein the patient is a geriatric patient.
